(19) United States
(12) Patent Application Publication
(10) Pub. No.: US 2026/0157840 A1
(43) Pub. Date: Jun. 11, 2026

(54) BIOINDUCTIVE ANCHOR AUGMENT FOR TISSUE REPAIR

(71) Applicant: Robert Trent McKay, Olympia, WA (US)

(72) Inventor: Robert Trent McKay, Olympia, WA (US)

(21) Appl. No.: 18/972,801

(22) Filed: Dec. 6, 2024

(51) Int. Cl. A61F 2/00; A61B 17/00; A61B 17/04; A61F 2/08

(52) U.S. Cl. CPC A61F 2/00; A61B 17/00; A61B 2017/0414; A61B 2017/0464; A61F 2002/0829

(57) ABSTRACT

A suture anchor augment includes middle and anchor portions configured to insert within an opening formed in bone of a patient, where it is wrapped around an anchor fixation device. The augment also features an outer or flag portion designed to remain external after insertion. One or more sutures are laced through the augment, which in part can be placed over and/or around tissue, allowing the suture to be passed through the tissue and knotted to secure it to the anchor fixation device. This bioinductive collagen conduit enhances the biological healing environment and is adaptable for various surgical applications, including tendon-to-bone repairs, ligament reconstructions, and other procedures requiring robust tissue integration. The anchor and middle portions may be infused with bone growth factors and the outer portion may be infused with healing factors to enhance tissue repair.

### FIELD OF THE INVENTION

This application relates to suture anchors for repairing tissue to bone interfaces. More specifically, this application pertains to the field of orthopedics, particularly the enhancement of existing bone fixation devices, such as suture anchors, used in tissue-to-bone interface repairs. The application finds relevance in various surgical applications, including but not limited to dental, plastic reconstructive, and pelvic floor repairs, providing an adaptable solution for any scenario involving the repair of tissue to bone.

### BACKGROUND OF THE INVENTION

When tissue is separated from bone by a traumatic injury it is common to use a suture anchor device to repair the tissue to the bone. These devices typically consist of an anchor portion, which provides stable fixation within the bone, and a suture portion that captures and secures the tissue to the bone. The anchor portion of the device provides a stable fixation point to the bone while the suture portion allows tissue to be captured. When the sutures are tied down around the tissue, it pulls the tissue into approximation with the bone and allows scar tissue to form a stable repair of the tissue to the bone. For example, common approaches utilize metal and synthetic materials to form an anchor device that is fixed within the bone.

Traditional suture anchors, both non-absorbable and absorbable, primarily focus on mechanical fixation and often overlook the biological aspects crucial for effective tissue repair. Existing devices are typically made of synthetic or metal materials, which do not promote biological communication between the bone marrow and the repair site—an essential factor for healing. Moreover, these anchors do not augment or restore the soft tissue component, which is frequently damaged during the initial injury.

### SUMMARY OF THE INVENTION

The present invention introduces a bioinductive collagen conduit designed to enhance the functionality of existing suture anchors. This conduit not only adapts to existing fixation devices but also provides a pathway for healing factors from the bone marrow to the repair site. Additionally, it augments the soft tissue component without compromising the mechanical fixation provided by the anchor.

In one aspect, the present invention relates to a bioinductive collagen conduit designed to augment existing suture anchor devices used for repairing tissue-to-bone interfaces. The collagen conduit is composed of a biocompatible, bioactive polymer, most commonly allograft collagen, which forms a sleeve that can be wrapped around standard suture anchors.

In one aspect, a suture anchor includes: an implanted portion configured to insert within an opening in bone of a patient; an exposed portion secured to the implanted portion configured to be exposed when the implanted portion is within an opening; and a suture laced through the exposed portion and the implanted portion such that tensioning of the suture will cause contraction of the implanted portion when the implanted portion is within the opening. The implanted portion and the exposed portion are formed of a biocompatible matrix for promoting tissue healing.

The device is configured with two primary portions: an implanted lower sleeve that secures the conduit within the bone by wrapping around the anchor, and an upper portion, referred to as the “flag,” that extends from the anchor insertion site into the repair zone. The lower sleeve provides an attachment of the device to the anchor, may enhance the fixation of the anchor within the bone, and provides a conduit for healing factors to access the upper portion of the device. The upper flag portion serves as a bioinductive pathway that facilitates the transfer of essential nutrients, growth factors, and cells from the bone marrow to the site of tissue repair. The upper portion is laced with the sutures from the standard suture anchor to provide further contact of the device with the anchor and further integrate into the repair. The implanted portion and the exposed portion may be formed from the same monolithic piece of bioactive polymer (such as allograft or electrospun collagen). The implanted portion and the exposed portion may be monolithically (e.g., as a single piece) formed of allograft collagen. The implanted portion and the exposed portion may be infused with one or more compounds to promote healing. The implanted portion may be infused with one or more compounds promoting bone growth, such as Bone Morphogenic Protein-2 (BMP-2) or similar growth factors to promote bone integration and secure the anchor more firmly within the bone. The exposed portion is infused with one or more growth factors, such as Platelet Rich Plasma (PRP). The conduit's upper flag portion is made of a bioactive polymer, typically collagen, that augments the soft tissue interface at the repair site. The flag portion can be infused with one or more biologically active compounds such as Platelet Rich Plasma (PRP) or growth factors, which are released into the repair site to enhance the healing process.

In some embodiments, the exposed portion and the implanted portion may be disposed adjacent to one another along a first direction, the implanted portion having a first width in a second direction perpendicular to the first direction, the exposed portion having a second width in the second direction that is greater than the first width. The second width may be at least three times the first width.

In some embodiments, the suture includes a first leg and a second leg, the first leg being laced through the exposed “flag” portion and the second leg being secured with the standard suture anchor.

In some embodiments, the suture anchor defines a proximal end defined by the exposed portion and a distal end defined by the implanted portion, the exposed portion being positioned between the implanted portion and the proximal end, the implanted portion being positioned between the distal end and the exposed portion. The suture may include a first leg and a second leg, the first leg being laced through the exposed portion and the implanted portion to an apex and the second leg being laced from the apex through the exposed portion only up to a position offset from the proximal end by at least 0.2 times a length of the suture anchor between the proximal end and the distal end. In some embodiments, the apex is offset from the distal end by less than 0.1 times the length of the suture anchor between the proximal end and the distal end.

In another aspect, a method includes forming an opening in a patient's bone and inserting an implanted portion of a suture anchor augment, which is wrapped around an anchor fixation device, such as a standard suture anchor, into the opening. The suture anchor augment includes an exposed portion that remains external to the bone after insertion, with a suture that is connected to the implanted anchor fixation device and laced through this exposed portion to secure it to the repair tissue. Once the suture anchor augment is wedged in the opening, the exposed portion of the suture may be passed through the tissue to be repaired and tied to secure the tissue to the traditional fixation device with the incorporated external portion of the anchor augment.

In some embodiments, the suture includes a first leg that arises from the suture anchor and laces through the exposed portion and a second leg extending from the apex. The method may further include passing the second leg through the tissue and tying a portion of the second leg extending from the tissue to the first leg. The exposed portion may be sized to cover the tissue. The tissue may be comprised of at least one of a tendon, ligament, or fascia.

The implanted portion may be infused with one or more compounds promoting bone growth and the exposed portion is infused with one or more growth factors. The one or more compounds promoting bone growth may include Bone Morphogenic Protein-2 (BMP-2) and the one or more growth factors may include Platelet Rich Plasma (PRP). The implanted portion and the exposed portion may be monolithically formed of allograft collagen.

This design provides a dual function: it not only reinforces the mechanical stability of the suture anchor but also enhances the biological healing environment by establishing a direct biological conduit from the bone marrow to the tissue being repaired. The collagen conduit is versatile and adaptable, allowing it to be used with various suture anchor designs without altering the surgeon's standard technique or implant choice. This ensures ease of integration into existing surgical practices while addressing complex clinical scenarios that involve tissue loss or compromised healing environments.

In another aspect, the invention allows for the augmentation of the soft tissue component in tendon-to-bone repairs, ligament reconstructions, and other procedures requiring robust tissue integration. The device can be modified in size and shape to accommodate different surgical needs, making it a highly flexible solution for a range of orthopedic and surgical applications.

### BRIEF DESCRIPTION OF THE DRAWINGS

Preferred and alternative examples of the present invention are described in detail below with reference to the following drawings:

FIG. 1A is a front view of a suture anchor having infused growth factors and healing factors in accordance with an embodiment of the present invention.

FIG. 1B is a front view of the suture anchor highlighting lacing of sutures through the suture anchor in accordance with an embodiment of the present invention.

FIG. 1C is a partial front view showing lacing of sutures in an implanted portion of the suture anchor in accordance with an embodiment of the present invention.

FIG. 1D is a front view showing a step in manufacturing the suture anchor in accordance with an embodiment of the present invention.

FIG. 1E is a front view showing an alternative step in manufacturing the suture anchor augment in accordance with an embodiment of the present invention.

FIGS. 1F-1G are front views of the anchor augment with sutures woven in an alternative embodiments of the present invention.

FIGS. 2A to 2D are side cross-sectional views showing the preparation of a site for receiving the suture anchor in accordance with an embodiment of the present invention.

FIGS. 3A to 3H are side cross-sectional views showing the use of a suture anchor to secure tissue to bone in accordance with an embodiment of the present invention.

FIGS. 3I to 3L are side cross-sectional views showing the use of a suture anchor augment to secure tissue to bone in accordance with an alternative embodiment of the present invention.

FIG. 4A is a side cross-sectional view of an alternative embodiment of a suture anchor to secure ligament to bone in accordance with an embodiment of the present invention.

FIG. 4B is a side view of yet another alternative embodiment of a suture anchor to secure ligament to bone in accordance with an embodiment of the present invention.

FIG. 4C is a side view of yet another alternative embodiment of a suture anchor augment to secure ligament to bone in accordance with an embodiment of the present invention.

FIGS. 5A to 5C are isometric views showing cartilage to bone repair in accordance with an embodiment of the present invention.

FIG. 5D is an isometric view showing cartilage to bone repair with suture anchor augment in accordance with an embodiment of the present invention.

### DETAILED DESCRIPTION OF THE PREFERRED EMBODIMENT

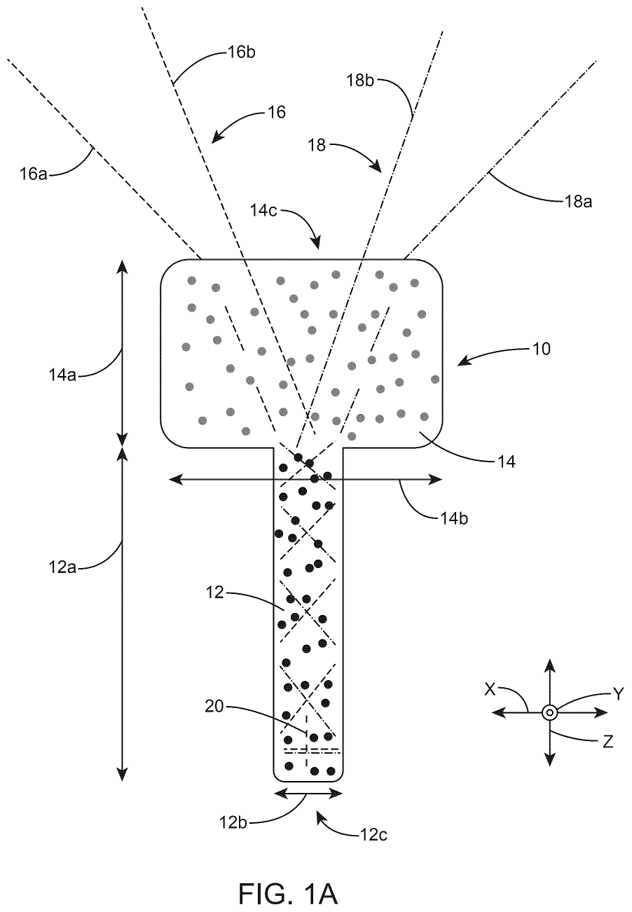

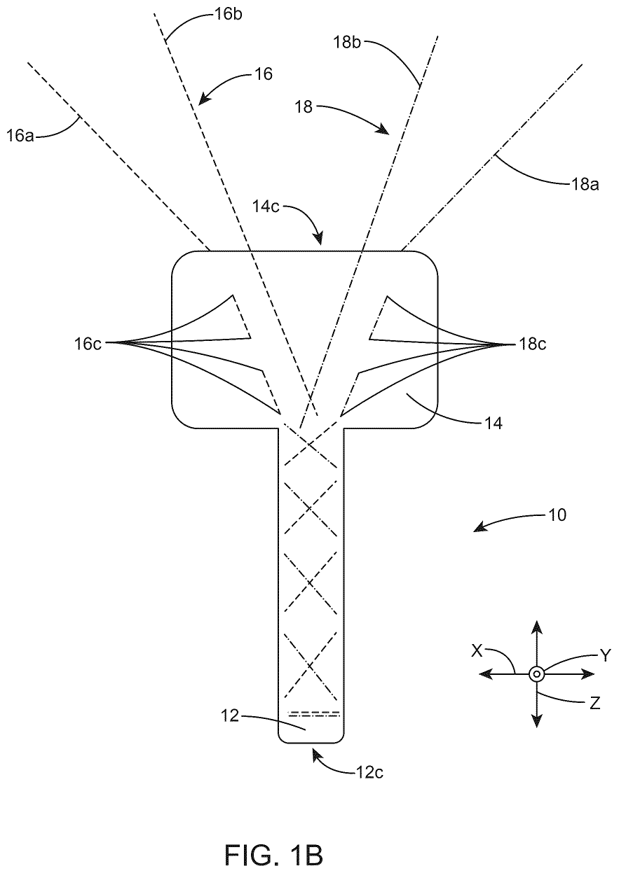

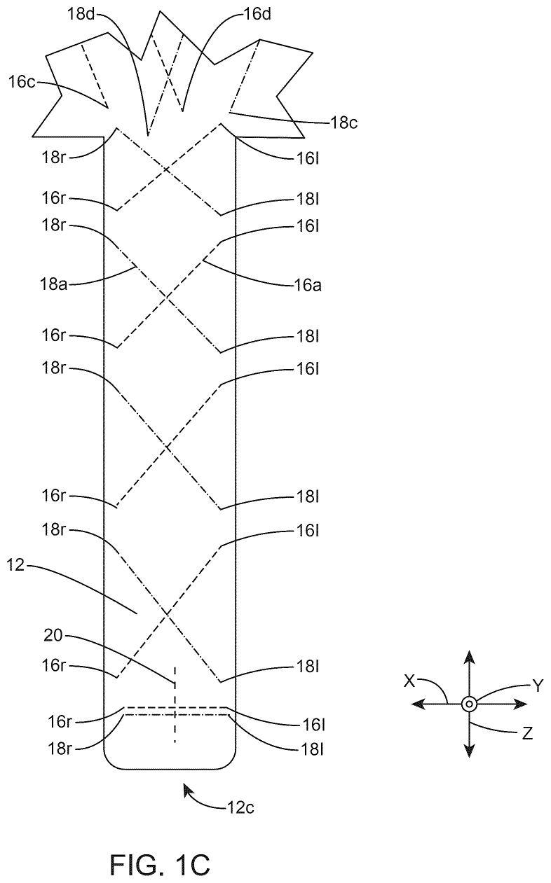

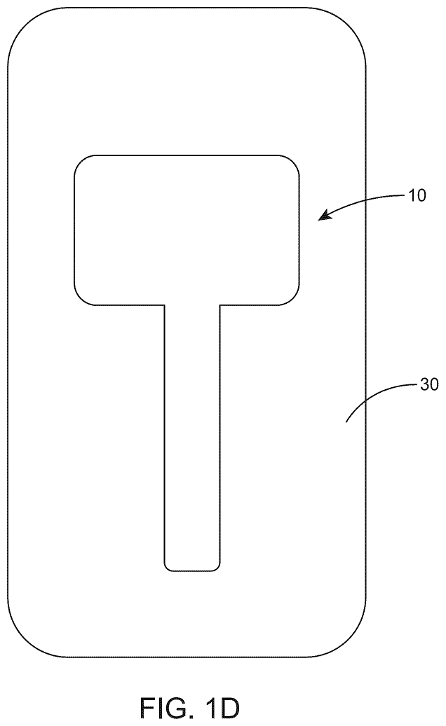

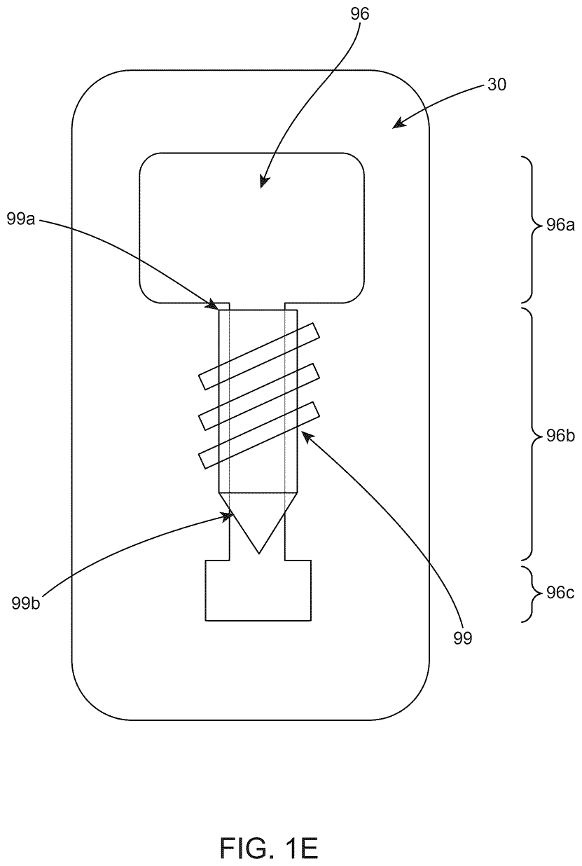

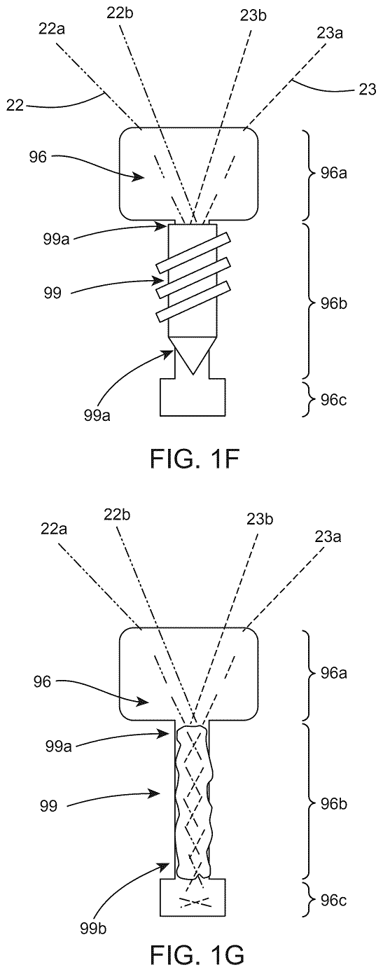

Referring to FIG. 1A, in prevalent orthopedic injuries, such as a rotator cuff tear, the tendon undergoes avulsion from its osseous insertion, specifically from the greater tuberosity of the proximal humerus. To remediate this, a surgical intervention is required to facilitate tissue-to-bone re-approximation. Conventionally, this is achieved utilizing suture anchor devices. These devices have a dual function: they provide osseointegration within the bone through an anchor and have affixed sutures to secure the avulsed tissue back to its anatomical position. Upon successful reattachment, osteotendinous integration ensues, characterized by the formation of fibrovascular scar tissue, which serves as the biomechanical interface, restoring functional integrity. This fibrovascular genesis is orchestrated by a cascade of cellular activities and the interplay of endogenous growth factors. The illustrated suture anchor 10 provides mechanical stability at the osteotendinous junction, fostering the conducive environment for healing. The suture anchor 10 may further augment this process by synergizing mechanical reattachment with a supplemental collagen matrix. This not only amplifies the scaffold for cellular migration but also establishes a bio-conduit, bridging the osseous compartment, a reservoir of autologous healing mediators, with the avulsed soft tissue. Additionally, this matrix can be imbued with exogenous bioactive agents to potentiate the regenerative environment. In the examples below, re-approximation of a tendon to bone using the suture anchor 10 is described. Of course, in other surgical instances, the tissue could represent other soft tissues such as ligaments, cartilage, fascia, skin, periodontal tissues, or organs. The disclosed suture anchor 10 may be used in the fields of orthopedic, dental, plastic, and reconstructive surgeries, as well as pelvic floor repairs. Without limitation, the disclosed suture anchor 10 may be deployed in any application where tissue is being repaired to bone or tissue requires implantation into bone for reconstruction.

The suture anchor 10 may be understood with reference to X, Y and Z directions that are mutually perpendicular. All references to dimensions and relative positions of the suture anchor 10 may be defined with the suture anchor 10 being undeformed and resting on a flat surface parallel to the X and Z directions.

The suture anchor 10 includes an implanted portion 12 and an exposed portion 14 disposed along the Z direction. All or part of the implanted portion 12 is positioned within an opening defined by bone during use whereas the exposed portion 14 remains external to the opening.

The implanted portion 12 has a length 12a in the Z direction and a width 12b in the X direction. The exposed portion 14 has a length 14a in the Z direction and a width 14b in the X direction. The thickness of the suture anchor 10 in the Y direction may be substantially uniform in the Y direction. As discussed in greater detail below, the implanted portion 12 inserts within an opening in bone and is collapsed to perform anchoring. The width 12b may therefore be smaller than the width 14b whereas the length 12a is greater than the length 14a. For example, the width 14b may be between 3 and 5 times the width 12b, between 4 and 4.6 times the width 12b, or between 4.2 and 4.4 times the width 12b. The length 12a may be between 1 and 3 times the length 14a, between 1.3 and 2.1 times the length 14a, or between 1.5 and 1.9 times the length 14a.

The implanted portion 12 and exposed portion 14 may have the illustrated rectangular shape, i.e., rectangles having the length and width 12a, 12b and the length and width 14a, 14b, respectively. The rectangular shapes may have rounded outer corners with rounded inner corners at the point of connection of the implanted portion 12 to the exposed portion 14. The corners may have, for example, a radius of curvature of between 0.1 and 0.5 times the width 12b. The corners may have equal or inequal radiuses of curvature.

The implanted portion 12 and the exposed portion 14 may be made of a collagen allograft material. Collagen allograft may be a biological material made of human acellular dermal matrix that closely replicates native human tissue and facilitates acceptance and incorporation of the suture anchor 10. Decellularized collagen matrix is readily available in multiple forms currently. The suture anchor 10 can be made of various other bioactive materials that are natural or synthetic (electrospun collagen, for example). The implanted portion 12 and the exposed portion 14 may also include different types of allograft tissue (Iliotibial (IT) Band, Achilles, etc.), acellular dermal matrix, or the like.

The implanted portion 12 and the exposed portion 14 may be made any of the natural degradable polymers, synthetic degradable polymers, and non-degradable polymers included in U.S. Pat. No. 9,974,534, which is incorporated herein by reference in its entirety.

The implanted portion 12 is placed in bone during use and may be infused with growth factors such as Bone Morphogenic Protein 2 (BMP-2) that facilitates rapid transformation of the collagen into bone. The exposed portion 14 is in contact with tissue to be secured to bone during use and may be infused with biologically active media such as independent growth factors or Platelet Rich Plasma (PRP) to improve the local healing environment and promote tissue repair. In the case of PRP, a sample could be obtained from the patient's blood pre- or intra-operatively, then infused into the exposed portion 14 prior to insertion. The PRP could then be stabilized by the collagen of the exposed portion 14 and kept in direct contact with the tissue being repaired, thereby increasing the healing capacity of the PRP at the interface to the tissue. The implanted and exposed portions 14, 12 are contiguous, which may facilitate the migration of growth factors that reside within the bone marrow to the tissue secured to the exposed portion 14, thereby further improving repair potential. The implanted and exposed portions 12, 14 may be treated with other compounds to promote healing, such as antibiotics, analgesics, anti-inflammatories, and/or matrix metalloproteinase inhibitors.

The implanted and/or exposed portions 12, 14 may be infused or coated with any of the biological, pharmaceutical, or other active ingredients listed in U.S. Pat. No. 9,981,061, which is hereby incorporated herein by reference in its entirety.

The sizes of the implanted and exposed portions 12, 14 can be variable depending on the intended application. The size of the exposed portion 14 relative to the implanted portion 12 may vary depending on an intended footprint of a repair. The exposed portion 14 may provide a “flag” of protruding tissue to be sutured to tissue with the implanted portion 12 being inserted within bone. The size and shape of the exposed portion 14 may therefore correspond to the tissue to be sutured thereto. Large repair defects (such as frequently seen in massive tears of the rotator cuff or chronic tendon lacerations) will utilize larger exposed portions 14. More limited applications will require smaller exposed portions 14. Unusually shaped defects could be repaired an exposed portions 14 cut in a non-rectangular shape conforming to the defect. In some embodiments, the suture anchor 10 could include hybrid materials, where the implanted portion 12 is made of one tissue or polymer, and the exposed portion 14 is made of a different tissue or polymer.

The suture anchor 10 may be used with one or both of a first suture 16 and a second suture 18, though in some applications a single suture 16 is used. Each suture 16, 18 may be high strength surgical suture, such as a surgical thread made of braided polyethylene. The suture 16, 18 preferably has sufficient strength to enable to the one or more sutures 16, 18 to draw the implanted portion 12 into an expanded ball thereby anchoring the suture anchor 10 into bone. As used herein, “anchoring” may refer to any wedging, balling up, catching, interference fit, or the like of the implanted portion with respect to an opening in which the implanted portion 12 is placed, as described in greater detail below.

Smaller tissue repair needs (such as hand surgery, cosmetic implants, dentistry) will utilize single suture, e.g., only one suture 16, and relatively smaller implanted and exposed portions 12, 14 (e.g., lengths 12a, 14a totaling between 5 and 15 millimeters). Larger tissue needs (shoulder or hip) can utilize larger upper and larger portions for use with two or more sutures 16, 18 (e.g., lengths 12a, 14a totaling between 15 and 40 millimeters).

The arrangement of the sutures 16, 18 relative to the suture anchor 10 may be understood with respect to a proximal end 14c and a distal end 12c of the suture anchor 10 that are positioned on opposite ends of the suture anchor 10 along the Z direction. The proximal end 14c may be defined as a point on the exposed portion 14 furthest from the implanted portion 12 along the Z direction and the distal end 12c may be defined as a point on the implanted portion 12 that is furthest from the exposed portion 14 along the Z direction.

Each suture 16, 18 includes a first leg 16a, 18a and a second leg 16b, 18b. The first leg 16a, 18a of each suture 16, 18 is laced through the exposed portion 14 and the implanted portion 12. The second leg 16b, 18b of each suture 16, 18 is laced through the implanted portion 12 and may also be laced through the exposed portion 14. The boundary between the first legs 16a, 18a and the second legs 16b, 18b (referred to herein as the “apex”) may be defined as the closest point to the distal end 12c at which the sutures 16, 18 cross a line 20 parallel to the Z direction and at a midpoint of the implanted portion 12 along the X direction.

Referring to FIG. 1B, the first leg 16a is laced through insertion points 16c in the exposed portion 14, the insertion point 16c closest to the proximal end 14c being separated from the proximal end 14c by a distance of between 0.02 and 0.10 times the total length of the suture anchor between the proximal end 14c and the distal end 12c (e.g., length 14a plus length 12a). The second leg 16b either (a) does not pass through any insertion points in the exposed portion 14 or (b) is passed through one or more insertion points 16d in the exposed portion 14 that are offset from the proximal end 14c by at least 0.1, 0.2, or 0.3 times the total length of the suture anchor 12 between the distal end 12c and the proximal end 14c.

The second leg 18a is laced through insertion points 18c in the exposed portion 14, the insertion point 18c closest to the proximal end 14c being separated from the proximal end 14c by a distance of between 0.05 and 0.25 times the length 14a. The second leg 18b either (a) does not pass through any insertion points in the exposed portion 14 or (b) is passed through one or more insertion points 18d in the exposed portion 14 that are at least 05, 0.7, or 0.9 times the length 14a from the proximal end 14c.

The insertion points 16c, 18c may define a tapered pattern: insertion points 16c, 18c further from the proximal end 14c along the Z direction may be closer to a center of the exposed portion 14 along the X direction than insertion points 16c, 18c that are closer to the proximal end 14c. The insertion points 16c, 18c may be aligned with one another along the Z direction or staggered slightly (e.g., between 0.01 and 0.05 times the length 14a). In the illustrated embodiments, the insertion points 16c are to the left of the insertion points 18c and the insertion points 16d are to the right of the insertion points 18d. In the illustrated embodiments, insertion points 18d, 16d are positioned inwardly from the insertion points 16c, 18c, e.g., the insertion points 16c, 18c furthest from the proximal end 14c.

As shown in FIG. 1B, the free ends of the first legs 16a, 18a may be extended from a first side of the exposed portion 14 whereas the free ends of the second legs 16b, 18b may be extend from a second side of the exposed portion 14 opposite the first side.

FIG. 1C illustrates the lacing of the legs 16a, 18a through the implanted portion 12. The suture 16 may be laced through the implanted portion 12 in a zig-zag pattern through left insertion points 16l and right insertion points 16r that are offset from one another in the X direction, such as by between 0.5 and 0.75 times the width 12b. The suture 18 may be laced through the implanted portion 12 in a zig-zag pattern through left insertion points 18l and right insertion points 18r that are offset from one another in the X direction, such as by between 0.5 and 0.75 times the width 12b. The insertion points 16l, 16r, 18l, 18r are distributed along the Z direction, such as to within 0.05 to 0.1 times the length 12a from the distal end 12c.

The leg 18b may extend from the apex 20, through an insertion point 18l or 18r and through the insertion point 18d without passing through any intervening insertion point. In other embodiments, the leg 18b is free and is not laced through the insertion point 18d or any other point on the implanted portion 12 or exposed portion 14. In still other embodiments, the leg 18b may be laced through the implanted portion 12 in a zig zag pattern, or a different pattern, as well as pass through the insertion point 18d.

In the illustrated embodiment, moving toward the distal end 12c, each right insertion point 16r, 18r is closer to the next insertion point 16l, 18l along the insertion direction than the insertion point 16l, 18l is to the next insertion point 16r, 18r. This uneven arrangement may facilitate curling of the implanted portion 12 when the sutures 16, 18 are tensioned as described in greater detail below.

At the apex, the sutures 16, 18 may extend across between insertion points 16l, 18l and insertion points 16r, 18r in a line substantially (e.g., within 15 degrees of) parallel to the X direction. The apex may be offset from the distal end 12c by less than 0.01, 0.02, 0.05, or 0.1 times the length of the suture anchor 10 between the distal end 12c and the proximal end 14c.

Referring to FIGS. 1C and 1D, the suture anchor 10 may be manufactured by cutting the suture anchor 10 from a piece 30 of collagen allograft material such that the implanted portion 12 and the exposed portion 14 are monolithically formed. Insertion points 16c, 16d, 16e, 16l, 16r, 18c, 18d, 18e, 18l, 18r may be cut as a separate manufacturing step or formed by a needle lacing the sutures 16, 18 through the suture anchor 10. The thickness of the piece 30 of collagen may vary based on the application, such as between 0.1 and 0.2 millimeters for hand and plastic surgery or from 2 to 3 millimeters for orthopedic surgery, such as hip and shoulder surgery. Embodiments will now address different applications of the invention, without limitation, to tendon (section A), ligament (section B), and cartilage (section C).

An alternative embodiment of the present invention showing the use of a suture anchor augment 96 to secure tissue to bone is described with reference to FIG. 1E to 1G. Referring to FIG. 1E, a suture anchor augment 96 may be manufactured by cutting an augment from a piece 30 of collagen allograft material, characterized with reference to three portions: an outer exposed or “flag” portion 96a, an implant spanning portion 96b, and a lower or anchor portion 96c. In this embodiment, the suture anchor augment 96 is described with reference to the three portions in relation to an anchor fixation device 99 having an inner end 99b to be inserted into an opening in a bone of a patient and an opposite outer end 99a. The anchor fixation device 99 may be one of many types of available anchor fixation devices to be used with the suture anchor augment 96 of the present invention; for example, metallic, plastic, polymeric, ceramic or other all-suture anchors. More specifically, the suture anchor augment 96 includes an outer exposed or flag portion that extends away from the outer end 99a of the anchor fixation device and may be cut to various sizes and shapes depending on application; an implant spanning portion 96b that forms the portion that spans the anchor fixation device 99 between the inner end 99b and the outer end 99a, and may be cut to various lengths depending on the size, length, composition, and fixation method of the anchor fixation device; and a lower or anchor portion 96c away from the inner end 99b of the anchor and that facilitates securing the anchor augment into the bone, which may be cut to various sizes and shapes depending on application. Preferably the three portions 96a, 96b, 96c are formed of a unitary materials or otherwise connected or secured to each other to form the anchor augment 96.

Referring to FIG. 1F, sutures 22 having legs 22a and 22b and 23 having legs 23a and 23b are woven through the exposed portion 96a of the anchor augment 96 to align and integrate the anchor augment 96 into the anchor fixation device 99. Referring to FIG. 1G, preferably when used with an all-suture anchor fixation device, sutures 22a and 23a are woven through the exposed portion 96a of the anchor augment, through the anchor fixation device 99 in the implant spanning portion 96b of the anchor augment, and through the anchor portion 96c of the anchor augment. Suture legs 22b and 23b are preferably placed through the repair tissue. This facilitates fully incorporating the anchor, augment, and sutures. As described in further detail below, the anchor is deployed and drawn up, the augment is drawn with it. The implant spanning portion 96b of the anchor augment 96b remains flexible and may be adjusted with respect to length, width, and thickness to accommodate the numerous available sizes, shapes, and functions of anchor fixation devices 99. This interplay between the sutures 22 and 23 is analogous to how sutures 16 and 18 are described above, save that in this embodiment sutures 22 and 23 arise from the anchor fixation device 99, whereas sutures 16 and 18 are incorporated into the anchor device and are critical to the drawing up of the anchored portion.

### Section A: Repair of Tendon to Bone

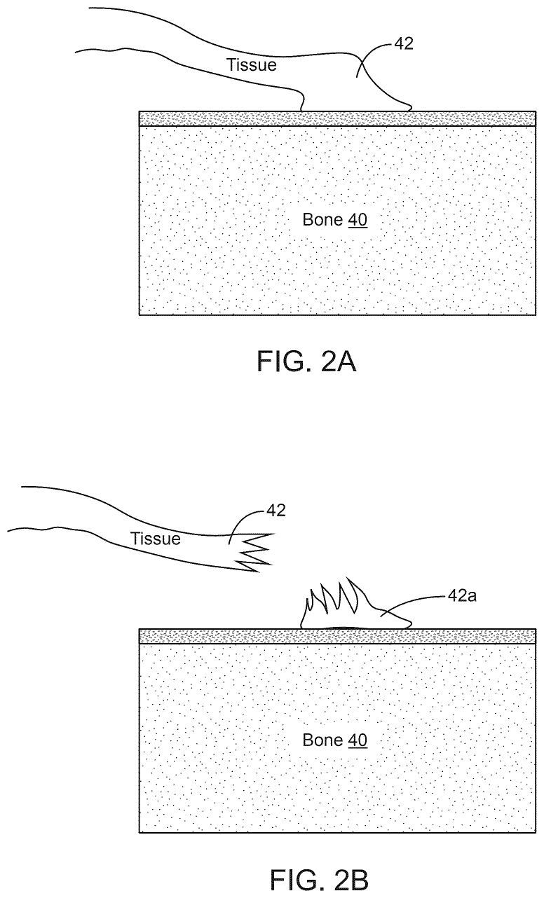

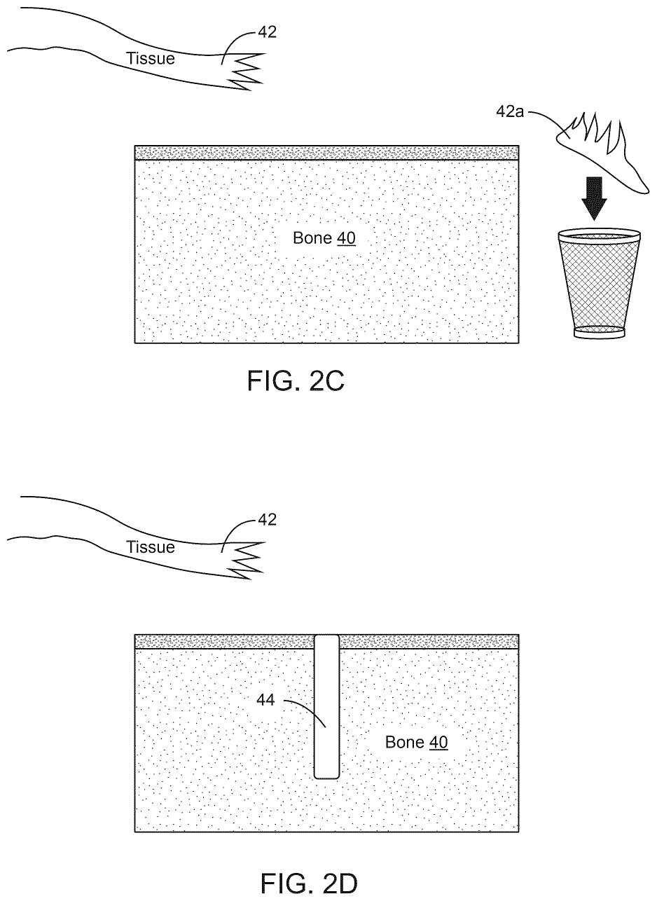

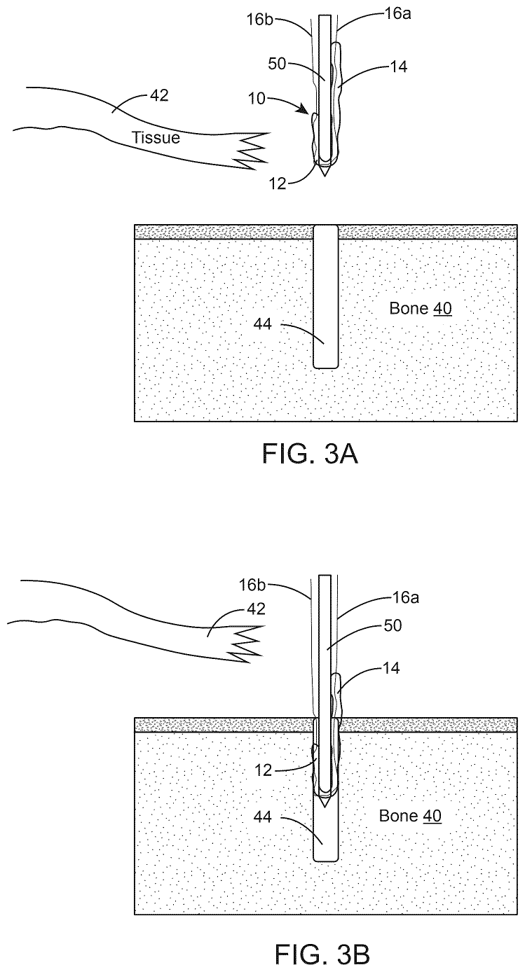

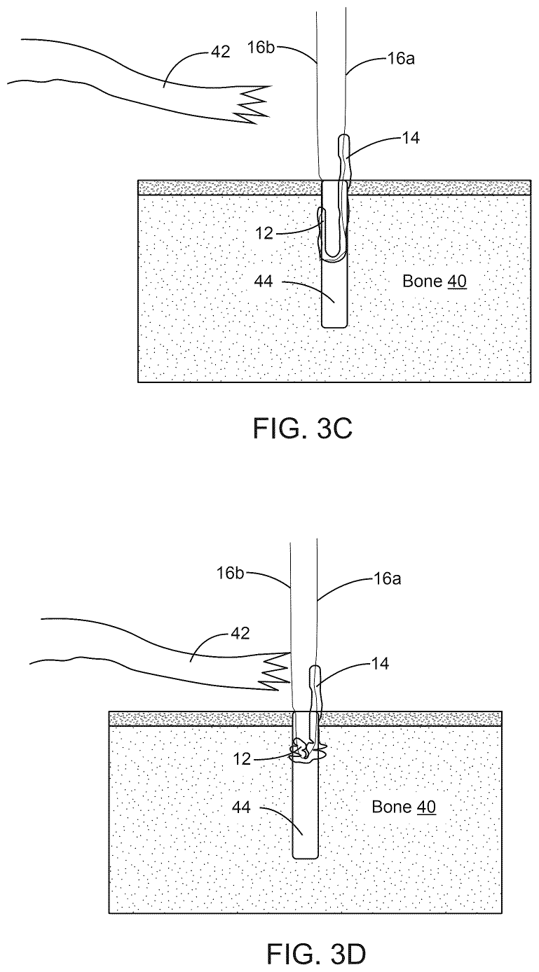

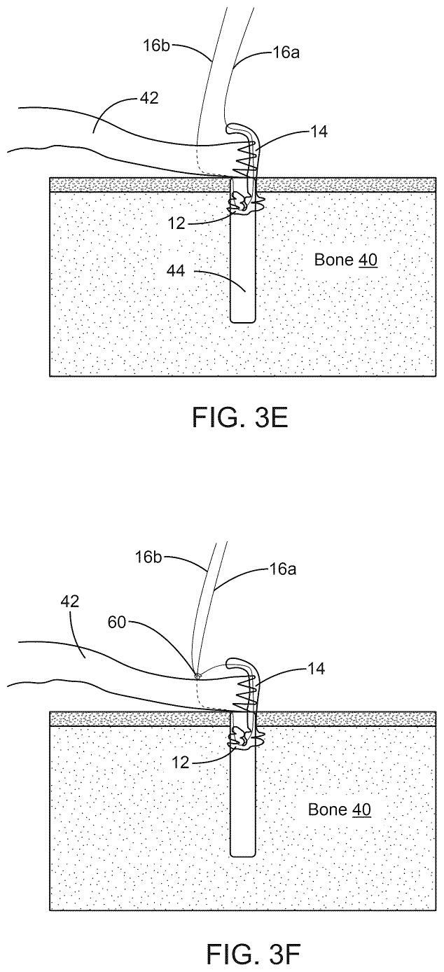

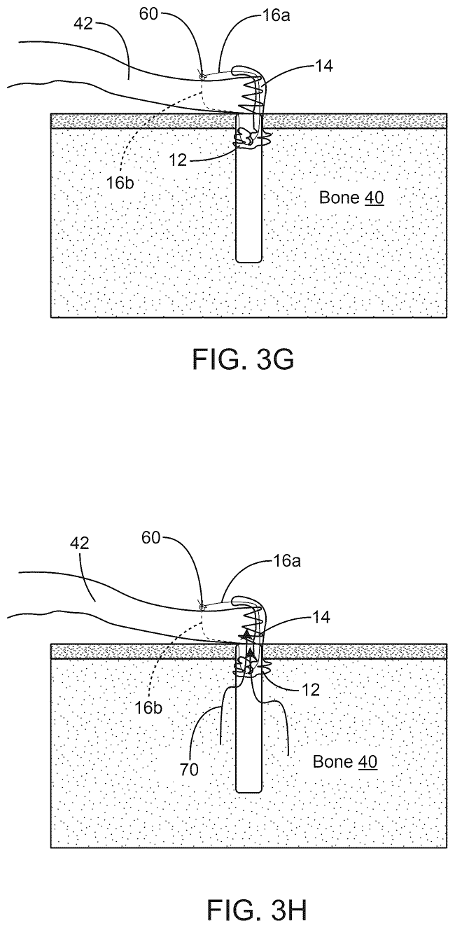

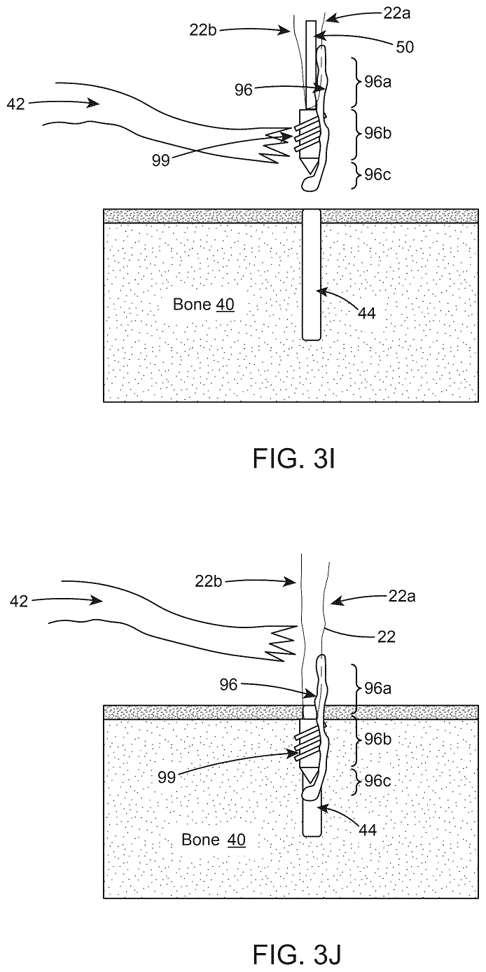

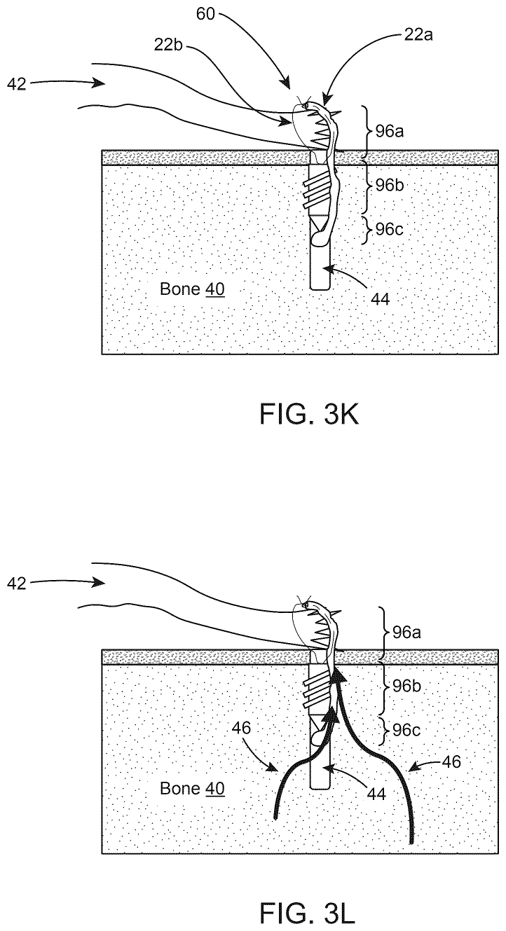

An example method of use of the suture anchor 10 is described below with respect to FIGS. 2A to 3H.

FIGS. 2A to 2D illustrate an example approach for preparing bone 40 for receiving the suture anchor 10. Referring specifically to FIG. 2A, prior to injury, tissue 42, such as a tendon, is secured to the bone 40. As shown in FIG. 2B, the tissue 42 may rupture, leaving a portion 42a of the tissue 42 secured to the bone. As shown in FIG. 2C, the portion 42a may be mechanically removed and discarded. Referring to FIG. 2D, a hole or opening 44 may be drilled in the bone 40 at or near the point where the portion 42a secured to the bone 40.

Referring to FIG. 3A, the suture anchor 10 may be draped over or inserted within an insertion tool 50. For example, the implanted portion 12 may be wrapped, wound, or otherwise engaged with the insertion tool 50. Alternatively, the insertion tool 50 may be positioned within a tube such that the insertion tool 50 is used to force the suture anchor 10 out of the tube. The insertion tool 50 may have a sharpened tip (radius of curvature less than 0.01 millimeter) such that the tool 50 partially penetrates the implanted portion 12. The exposed portion 14 may be positioned along the insertion tool 50. The legs 16a, 16b of the suture 16 may also extend along the insertion tool and away from the tip of the insertion tool 50. In the examples of FIGS. 3A to 3H, a single suture 16 is shown with the understanding that a second suture 18 would function in a like manner.

Referring to FIG. 3B, the insertion tool 50 and at least part of the implanted portion 12 are inserted within the opening 44. The legs 16a, 16b extend outwardly from the opening 44 following insertion with all or part of the exposed portion 14 extending outwardly from the opening 44. The larger width 14b of the exposed portion 14 may resist insertion of the exposed portion 14 into the opening 44. For example a diameter of the opening 44 (e.g., at the surface of the bone 40) may be less than 0.8, less than 0.6, or less than 0.5 times the width 14b.

Referring to FIG. 3C, the insertion tool 50 may then be withdrawn from the opening 44, leaving at least a portion of the implanted portion 12 within the opening 44. The implanted portion 12 may engage walls of the opening 44 to resist removal as the insertion tool 50 is withdrawn. For example, the width 12b of the implanted portion 12 may be greater than 1.05, 1.1, or 1.25 times the diameter of the opening 44 (e.g., at the surface of the bone 40).

Referring to FIG. 3D, one or both of the legs 16a, 16b may then be tensioned. The tension on one or both of the legs 16a, 16b along with friction between the implanted portion 12 and the opening 44 will cause the implanted portion 12 to bunch and/or curl within the opening 44. The bunching and/or curling of the implanted portion 12 causes the implanted portion 12 to become wedged within the opening 44.

Referring to FIG. 3E, a free end of one or both of the legs may then be inserted through the tissue 42. For example, the leg 16b may be suited for this task due to being laced through less of the exposed portion 14. One or both of the legs 16a, 16b, such as the leg 16b passing through the tissue 42, may be precoated with collagen to promote healing and bonding with the tissue 42.

As shown in FIG. 3F, The legs 16a, 16b of the suture 16 may then be tied to one another, such as using an appropriate surgical knot 60. As the legs 16a, 16b are tied to one another, the legs 16a, 16b may press the exposed portion 14 against the tissue 14. For example, the threading of the leg 16a through the exposed portion 14 may cause the leg 16a to pull the exposed portion 14 against the tissue 42 when knotted with the leg 16b. The free ends of the legs 16a, 16b may then be cut as shown with reference to FIGS. 3G to 3H.

An alternative embodiment of the present invention showing the use of a suture anchor augment to secure tissue to bone is described with reference to FIGS. 3I to 3L. Referring to FIG. 3I, suture leg 22a is incorporated into flag portion 96a of suture anchor augment 96. An insertion tool 50 is used to force the anchor fixation device 99 and at least portions of the suture anchor augment 96 within opening 44, preferably including the insert spanning portion 96b and the anchor portion 96c, which wraps around the inner end 99b of the anchor fixation device 99 to facilitate centering the anchor augment 96 while it is being inserted, leaving the flag portion 96a protruding outside opening 44 for incorporation into the soft tissue portion of the repair.

Referring to FIG. 3J, one or both of the suture legs 22a, 22b may then be tensioned. Leg 22a is preferably woven into the flag portion 96a of the anchor augment while leg 22b is kept free. The tension on one or both of the legs 22a, 22b along with friction between the suture 22, anchor augment 96, and anchor fixation device 99 against the opening 44 will cause at least part of the insert spanning portion 96b of the anchor augment and the anchor portion 96c of the anchor augment 96 to become wedged between opening 44 and the anchor fixation device 99. The insertion tool 50 is removed leaving the anchor fixation device 99 with insert spanning portion 96b and anchor portion 96c of the anchor augment 96 in the bone 40 and the suture leg 22a integrated with the flag portion 96a and suture leg 22b exposed to secure the device to the target tissue 42.

Referring to FIG. 3K, a free end of one or both of suture legs 22a,b may then be inserted through the tissue 42. One or both of the legs 22a,22b, such as the leg 22b passing through the tissue 42, may be precoated with collagen to promote healing and bonding with the tissue 42. The legs 22a,22b of the suture 22 may then be tied to one another, such as using an appropriate surgical knot 60. As the legs 22a, 22b are tied to one another, the legs 22a, 22b may press the flag portion 96a of the anchor augment against the tissue 42. For example, the threading of the leg 22a may cause the flag portion 96a of the anchor augment to contact the tissue 42 when knotted with the leg 22b. As shown with reference to FIG. 3L, because the bone marrow naturally produces healing factors, the interconnected flag, insert spanning and anchor portions of the anchor augment—which are now in contact with both the bone 40 and tissue 42—allow the factors to wick through the collagen in the anchor augment from bone to tissue (see reference paths 46) into the repair to provide an improved healing environment. The free ends of the legs 22a,22b may then be cut as shown with reference to FIGS. 3G to 3H.

Once the suture 22 is knotted, the collagen thereof is automatically woven into the repair, thereby enhancing the interface with additional collagen. A major benefit of the collagen allograft material is the pliability thereof, which allows the collagen of the exposed portion 96a to conform to the tissue 42 and fills defects that may be left due to trauma the tissue may have received during the injury. The additional collagen structure of the flag portion 96a of the anchor augment 96 further mimics the natural flare of tendons, enhancing healing and the ultimate mechanical strength of the repair. The suture 22 (and possibly the additional suture 23) aids in fixing the tissue 42 to the bone 40, promoting tissue ingrowth and repair. The anchor fixation devices available typically include 1-3 sutures emanating from the top of the device, such as the embodiment shown herein where sutures 22 and 23 describe a two-suture version. But an anchor fixation device may include more, for example as shown in other embodiments where five different sutures are shown. Regardless the number of sutures, preferably all of the sutures from the device are incorporated into the augment. In the drawing this represents 22 and 23 for a two suture version.

Following treatment, healing factors 70 migrate from the bone 40 into the implanted portion 12. Some of the healing factors 70 will further migrate into the exposed portion 96a and facilitate healing of the tissue 42 and binding of the tissue to the bone 40 and to the flag portion 96a.

In the illustrated examples, the suture anchor augment 96 is used for a rotator cuff repair, which is illustrated in FIGS. 3A to 3L. For example, the suture anchor augment 96 may be used in single-row rotator cuff repairs. In such applications, the opening 44, implant spanning portion 96b, and anchor portion 96c may be placed in approximately (e.g., within 3 millimeters of) the middle of the greater tuberosity of the proximal humerus

The method of use of the suture anchor augment 96 described above with respect to FIGS. 2A to 3L may also be used in at least the following applications that are included here by way of example and not limitation.
Patellar Tendon Repair
Quadriceps Tendon Repair
Achilles Tendon Repair
Proximal Hamstring Repair
Distal Biceps Tendon repair
Finger Flexor Tendon Repair
Triceps Avulsion repair
Pectoralis Tendon rupture repair

### Section B: Ligament to Bone Repairs

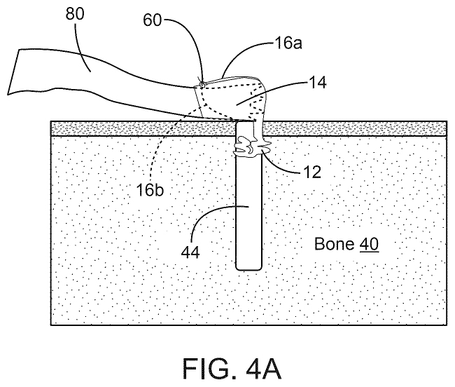

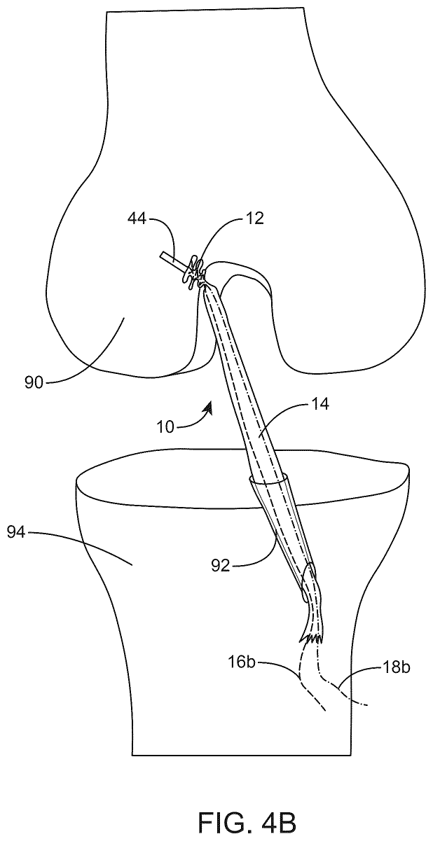

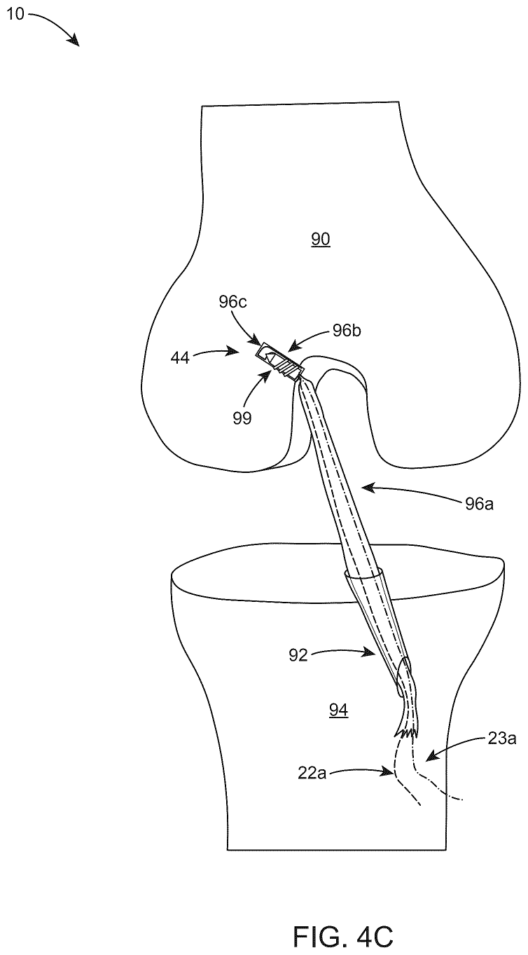

Referring to FIG. 4A in another example, the suture anchor 10 may be used to perform an Anterior Cruciate Ligament reconstruction. An Anterior Cruciate Ligament (ACL) rupture is the disruption of a ligament 80 that attaches the femur to the tibia within the knee and is critical to knee stability. Often the injury causes severe irreparable destruction of the tissues that must be reconstructed from other tissue. In accordance with the process shown in FIGS. 2A to 3H, the destroyed remnants of the native ACL are either removed or incorporated into the reconstruction. The implanted portion 12 of the suture anchor 10 may be implanted in an opening 44 in the femoral footprint of the original torn ligament 80. The exposed portion 14 of the suture anchor 10 may be made, in at least this example, as a long tubular portion of collagen that emulates the size of the torn ligament 80 of the recipient. The implanted portion 12 may be wedged within the opening 44 as described above with respect to FIGS. 3A to 3D and the torn ligament 80 may be inserted within the tubular exposed portion 14 and secured thereto using the suture 16, e.g., passing leg 16b through the tubular exposed portion 14 and the torn ligament 80. This provides a strong, stable fixation into the tibia and a large collagen graft to extend through the joint. The end of the exposed portion 14 may additionally or alternatively be secured into the tibia using other fasteners, such as an interference screw passing through the tubular exposed portion 14 and into the ligament.

FIG. 4B is another example of the use of the suture anchor 10 to perform ACL reconstruction. The opening 44 may be formed in the femur 90, such as at or near (e.g., within 5 millimeters of) a former point of attachment of the ACL and the implanted portion 12 may be secured in the opening 44 as described above. The exposed portion 14 may be particularly long, e.g., 2, 5, 10, or more times longer than the implanted portion 12. The exposed portion 14 may be passed through a channel 92 formed in the tibia 94 and be secured therein or elsewhere on the tibia using another fastening approach, including the use of another suture anchor 10 to secure to the exposed portion 14 in lieu of the tissue 42 in the examples above. In another example, the exposed portion 14 may be configured to wedge within the channel 92 similar to the implanted portion 12. In the embodiment of FIG. 4B, the exposed portion 14 may serve as a prosthetic ACL rather than securing to a remnant of the ACL as for the case of FIG. 4A.

FIG. 4C is an alternative embodiment using a suture anchor augment 96 to perform ACL reconstruction. The opening 44 may be formed in the femur 90, such as at or near (e.g., within 5 millimeters of) a former point of attachment of the ACL, and at least part of the insert spanning portion 96b of the anchor augment and the anchor portion 96c of the anchor augment will be implanted into and become wedged within the opening 44 using an anchor fixation device 99, as described above with reference to FIGS. 3I to 3L. In this embodiment, the flag portion 96a of the anchor augment may be particularly long, e.g., 2, 5, 10, or more times longer than the insert spanning portion 96b and the anchor portion 96c of the anchor augment. The flag portion 96a of the anchor augment may be passed through a channel 92 formed in the tibia 94 and be secured therein or elsewhere on the tibia using another fastening approach such as an interference screw, Endo button, or being tied over a post. In another example, the flag portion 96a may be configured to wedge within the channel 92 similar to how the anchor augment 96 was implanted in opening 44. In the embodiment of FIG. 4C, the anchor augment 96 may serve as a prosthetic for tissue reconstruction rather than securing to a remnant of tissue as for the case of FIG. 4A.

The approach of FIGS. 4A to 4C may be used in a similar manner to perform other common ligament to bone repairs or reconstructions, such as any of the following applications that are listed here by way of example and without limitation:
Posterior Cruciate Ligament Repairs/Reconstruction
Medial or Lateral Collateral Ligament Repairs/Reconstructions
Thumb Ulnar Collateral ligament reconstruction
Elbow Lateral Ulnar Collateral Ligament Repairs/Reconstruction
Ankle Anterior Talofibular ligament Repairs/Reconstructions
Plantar plate Repair/Reconstructions

### Section C: Cartilage to Bone Repairs

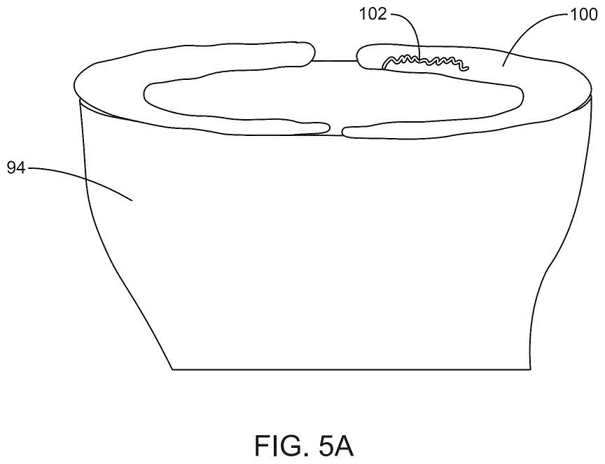

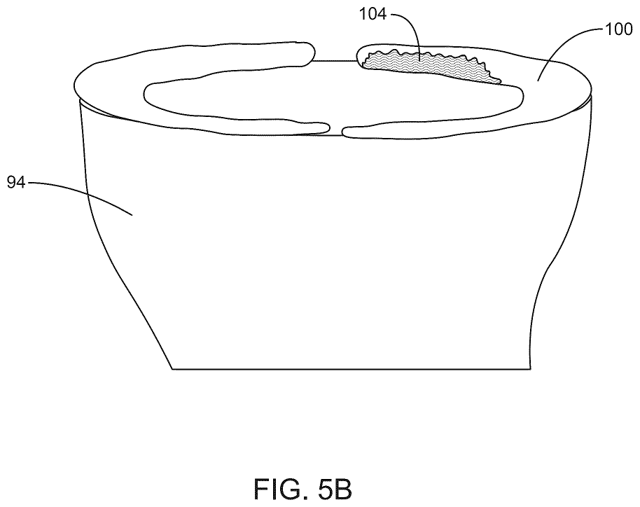

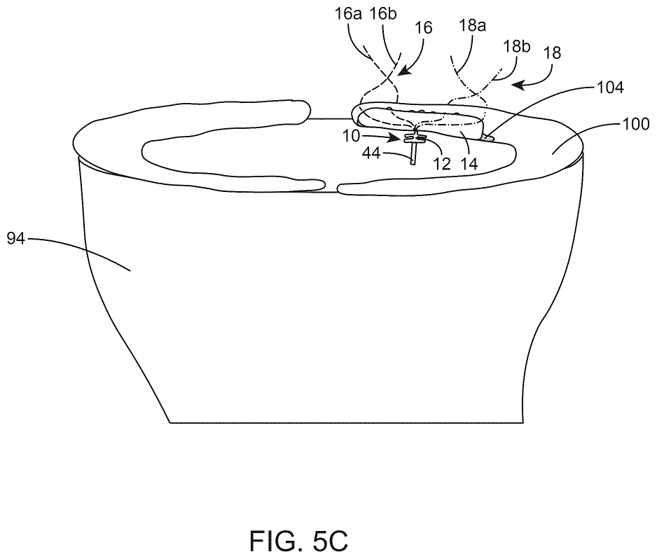

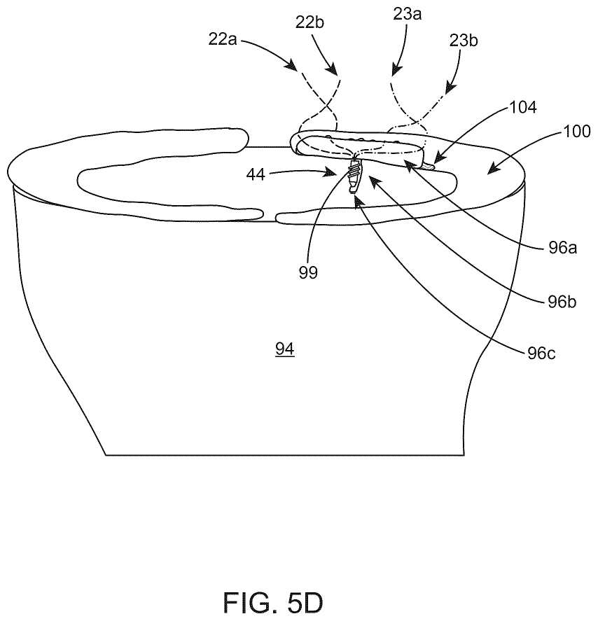

The approach of any of FIG. 2A to 3I may be used in a similar manner to perform other common cartilage to bone repairs such as meniscal repair or reconstruction. For example, referring to FIG. 5A, the meniscus 100 is a ring of collagen that attaches to the proximal tibia within the knee with multiple functions including shock absorber and knee stabilizer. When a tear 102 is formed in the meniscus 100, the tissue of the meniscus is often not repairable and leads to a deficiency of tissue.

Referring to FIG. 5B, an area 104 of the meniscus 100 around the tear 102, such as between the tear 102 and the tibia 94 may be removed to create a cleared area 104.

Referring to FIG. 5C, the implanted portion 12 of the suture anchor 10 may be deployed into an opening 44 formed in the bed of the meniscal attachment within the proximal tibia 94. The exposed portion 14 in this example may be shaped to emulate the shape of the meniscus, which may enable the exposed portion 14 to be incorporated into a repair to surrounding tissues. In particular, the exposed portion 14 may be much wider than the implanted portion 12 in this example, such as 5, 10, 20, or more times wider in order to extend over substantially all (e.g., at least 70, 80, or 90 percent) of the cleared area 104. The sutures 16, 18 may then secure to the remaining portion of the meniscus 100 in the same manner as described above. For example, the legs 16b, 18b may be passed through the remaining portion of the meniscus 100 and knotted to the legs 16a, 18a, respectively. The points of insertion of the legs 16b, 18b may extend outwardly on either side of the implanted portion 12 to provide a stable and distributed attachment of the exposed portion 14 to the remaining portion of the meniscus 100.

Referring to FIG. 5D, at least part of the insert spanning portion 96b of the anchor augment and the anchor portion 96c of the anchor augment will be implanted into and become wedged within the opening 44 formed in the bed of the meniscal attachment within the proximal tibia 94 using an anchor fixation device 99, as described above with reference to FIGS. 3I to 3L. The flag portion 96a in this example may be shaped to emulate the shape of the meniscus, which may enable the flag portion to be incorporated into a repair to surrounding tissues. In particular, the flag portion 96a may be much wider than the insert spanning and anchor portions 96b, 96c in this example, such as 5, 10, 20, or more times wider in order to extend over substantially all (e.g., at least 70, 80, or 90 percent) of the cleared area 104. The sutures 22,23 may then secure to the remaining portion of the meniscus 100 in the same manner as described above. For example, the legs 22b,23b may be passed through the remaining portion of the meniscus 100 and knotted to the legs 22a,23a, respectively. The points of insertion of the legs 22b,23b may extend outwardly on either side of the middle and anchor portions to provide a stable and distributed attachment of the flag portion 96a to the remaining portion of the meniscus 100.

Other common ligament to bone repairs that may be completed in a similar manner, such as Shoulder Labral repair (Bankart, SLAP, posterior labral repair), Wrist Triangular Fibrocartilage (TFCC) repair, and Hip labrum repair/reconstruction.

In another embodiment, urinary incontinence is treated with a sling procedure where graft tissue or mesh is utilized to reinforce the neck of the bladder. The implanted portion 12 of the suture anchor 10 or suture anchor augment 96 described above may be wedged in an opening 44 in the pubic bone on each side of the bladder. Depending on the embodiment, the exposed portion 14 of the suture anchor 10 or suture anchor augment 96 may then be wrapped around the neck of the bladder and sutured in place to provide the tensioning sling.

The suture anchor augment 96 and corresponding methods of use may provide at least the following advantages may also be used in at least the following applications that are included here by way of example and without limitation:
A biological scaffold for tissue repair.
Faster and superior tissue healing potential due to the biological nature of the material.
Collagen providing a bio-inductive medium that transports growth factors from the bone marrow into the exposed portion 96a of the repair.
Allograft collagen exposed portion 96a reforms the tendon, which is often heavily traumatized, and fills in tissue defects.
A simple, single-step process that provides insertion and fixation of an anchor in bone, suture for repair of tissue to the bone, allograft biopolymer to enhance the repaired tissue, a conduit for repair proteins from the bone marrow, and a scaffold for healing factors.
The preferred fixation device utilized by the proceduralist can be retained while allowing a collagen allograft that incorporates into the fixation without further instrumentation or training.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A suture anchor augment for use with an anchor fixation device having an inner end and an outer end, comprising:
an anchor portion extending away from the inner end of the anchor fixation device, configured to insert within an opening in a bone of a patient;
a middle portion secured to the anchor portion and extending between the inner and outer ends of the anchor fixation device, configured to insert within the opening;
an outer portion extending away from the outer end of the anchor fixation device, configured to be exposed outside of the bone of the patient when the anchor and middle portions are within the opening; and
a suture laced through at least a part of one of the outer, middle, and inner portions such that tensioning of the suture will cause contraction of at least one of the anchor or middle portions when the anchor and middle portions are within the opening.

2. The suture anchor augment of claim 1, wherein the anchor, middle and outer portions are monolithically formed of bioactive polymer.

3. The suture anchor augment of claim 1, wherein the anchor, middle and outer portions are monolithically formed of allograft collagen.

4. The suture anchor augment of claim 1, wherein at least one of the anchor and middle portions are infused with one or more compounds to promote bone growth.

5. The suture anchor augment of claim 4, wherein the one or more compounds include Bone Morphogenic Protein-2 (BMP-2).

6. The suture anchor augment of claim 1, wherein the outer portion is infused with one or more compounds to promote healing.

7. The suture anchor augment of claim 6, wherein at least one or more compounds include Platelet Rich Plasma (PRP).

8. The suture anchor augment of claim 1, wherein the outer portion and the middle portion are disposed adjacent one another along a first direction, the middle portion having a first width in a second direction perpendicular to the first direction, the outer portion having a second width in the second direction that is greater than the first width.

9. The suture anchor augment of claim 8, wherein the second width is at least as large as the first width.

10. The suture anchor augment of claim 1, wherein the suture includes a first leg and a second leg, the first leg being laced through the outer portion and the anchor portion to an apex and the second leg being laced through the anchor fixation device.

11. The suture anchor augment of claim 10, wherein the apex is offset from the inner end by less than 0.1 times the length of the anchor fixation device between the inner end and the outer end.

12. A method comprising:
forming an opening in a bone of a patient;
inserting at least part of a suture anchor augment into the opening, wherein:
the suture anchor augment includes an anchor portion, a middle portion integrated with an anchor fixation device, and an outer portion, and
the outer portion is configured to be exposed outside of the opening following insertion of the anchor and middle portions;
lacing a suture through at least a part of at least one of the outer, middle and anchor portions; and
passing the suture through tissue being repaired and tying the suture to secure the tissue to the suture anchor.

13. The method of claim 12, wherein the suture includes a first leg laced through the outer and anchor portions to an apex and a second leg extending from the apex and integrated with the anchor fixation device, further comprising passing the second leg through the tissue and tying a portion of the second leg extending from the tissue to the first leg.

14. The method of claim 12, wherein the outer portion is sized to cover the tissue.

15. The method of claim 14, wherein the tissue is at least one of a tendon, ligament, or fascia layer.

16. The method of claim 12, wherein at least one of the anchor and middle portions are infused with one or more compounds promoting bone growth.

17. The method of claim 16, wherein the one or more compounds include Bone Morphogenic Protein-2 (BMP-2).

18. The method of claim 12, wherein the outer portion is infused with one or more compounds to promote healing.

19. The method of claim 18, wherein the one or more compounds includes Bone Morphogenic Protein-2 (BMP-2).
